(19) United States
(12) Patent Application Publication
(10) Pub. No.: US 2008/0233221 A1
(43) Pub. Date: Sep. 25, 2008

(54) Prostate cancer and benign prostatic hyperplasia treatments

(75) Inventors: Emily Ho, Corvallis, OR (US); Jan Frederik Stevens, Corvallis, OR (US); Cristobal L. Miranda, Corvallis, OR (US); Donald Buhler, Corvallis, OR (US); Max L. Deinzer, Corvallis, OR (US)

Correspondence Address: KLARQUIST SPARKMAN, LLP, 121 SW SALMON STREET, SUITE 1600, PORTLAND, OR, 97204, US

(73) Assignee: State of Oregon

(21) Appl. No.: 11/728,228

(22) Filed: Mar. 23, 2007

(51) Int. Cl. A61K 36/899; C12N 5/06

(52) U.S. Cl. 424/750; 435/375

(57) ABSTRACT

The present invention provides methods of treating prostate cancer and benign prostatic hyperplasia using xanthohumol and/or a xanthohumol analog.

### ACKNOWLEDGMENT OF GOVERNMENT SUPPORT

This work was supported by a grant from the National Institute of Health NIH CA107693 and a grant from National Institute of Environmental Health Sciences Center P30ES00210. The United States Government has certain rights in this invention.

### BACKGROUND OF THE INVENTION

Xanthohumol (XN) is the principal flavonoid found in the hop plant, Humulus lupulus L. (Cannabaceae). Hops are traditionally used to add bitterness and flavor to beer. More recently, however, alternative uses for hop compounds and their effects on biological processes have become an area of interest. For example, studies have indicated that xanthohumol exhibits antioxidant and free radical scavenging properties.

Prostate cancer remains a considerable health problem for men around the world, accounting for an anticipated 30,000 deaths in 2005 in the United States alone. In fact, prostate cancer is the most frequently diagnosed non-cutaneous cancer and is the second leading cause of cancer death in American men. With little progress being made in reduction of these rates, identification and utilization of novel compounds for cancer prevention has become an important issue in public health related research.

Benign prostatic hyperplasia (BPH), a disease in which prostate epithelial cells grow abnormally and block urine flow, afflicts more than 10 million adult males in the United States, and many millions more throughout the rest of the world. Until relatively recently, surgical intervention was the only treatment of the disease, and even today, surgery is the treatment of last resort, almost inevitably relied upon when other treatments are not, or cease to be, effective. Prostate surgery and recovery therefrom is painful, and the surgery itself may not be effective and poses the risk of serious side effects.

Control of cell cycle and apoptosis are key mechanisms linked to the suppression of cell proliferation in several chemopreventive agents. In addition, inhibition of NFkB activation has been another key chemoprevention target. Constitutive activation of NFkB is common in various human malignancies, including prostate and leads to up-regulation of genes encoding adhesion molecules, inflammatory cytokines, growth factors, and anti-apoptotic genes.

There is a need in the art for novel and effective treatments of prostate cancer and BPH. The present invention addresses these and other needs in the art.

### BRIEF DESCRIPTION OF THE DRAWINGS

FIG. 1 sets forth the chemical structures of xanthohumol (XN) and xanthoaurenol (XAL).

FIG. 2 sets forth data relating to xanthohumol and xanthoaurenol inhibition of cellular proliferation of benign prostate hyperplasia (BPH-1) and cancerous PC-3 cells. BPH-1 cells were treated with (A) epicatechin (EC), (B) epigallocatechin gallate (EGCG), (C) xanthohumol (XN) or (D) xanthoaurenol (XAL) for 48 h. In (E) and (F), androgen-independent cancerous PC-3 cells were treated with xanthohumol (XN) and xanthoaurenol (XAL). Viability was determined using the MTT assay. Values are mean±SD, n=4.

FIG. 3 sets forth data relating to xanthohumol and xanthoaurenol induction of caspase activity in BPH-1 and PC-3 cells. Cells were treated with XN or XAL for 48 h. Induction of apoptosis was measured using a multi-caspase assay. SR-VAD-FMK covalently binds caspases and positively stained cells were assessed by flow cytometry as a measure of caspase activation. 7-Amino-actinomycin D (7AAD), a nucleotide stain that only stains cells when membrane integrity is compromised, was used to differentiate between early and late apoptotic cells. SR-VAD-FMK(+) 7AAD(−) cells are undergoing early-mid apoptosis, SR-VAD-FMK(+) 7AAD(−) represent late stage apoptotic cells. Values are mean±SD, n=3. *P<0.05, **P<0.01.

FIG. 4 sets forth data relating to xanthohumol and xanthoaurenol induction of apoptosis in BPH-1 and PC-3 cells. Cells were treated with XN and XAL and apoptosis was detected using the Annexin V assay. Externalization of phosphatidyl serine, an early marker of apoptosis was detected by flow cytometry after staining with Annexin V-PE and 7AAD. Annexin V(+) 7AAD(−) cells are undergoing early apoptosis, and Annexin V(+) 7AAD(+) are undergoing late apoptosis. Values are mean±SD, n=3. *P<0.05, **P<0.01.

FIG. 5 sets forth data relating to xanthohumol and xanthoaurenol inhibit NFκB activation in nuclear extracts of BPH-1 cells. Cells were treated with XN or XAL for 48 h. (A) Cells were harvested by scraping and then subjected to nuclear extraction. NFκB activation was determined using transcription factor binding ELISA as described in Section 2. Equal protein loading was confirmed using β-actin. Results are representative of two or more separate experiments. (B) BPH-1 cells were treated with XN or XAL for 48 h and detection of p65 nuclear protein was performed with immunoblotting methods. (C) No inhibition of NFκB activation in treated PC3 cells. Results are mean±SD, n=3. **P<0.01.

FIG. 6 sets forth data relating to xanthohumol and xanthoaurenol induce expression of proapoptotic proteins p53 and bax, with down-regulation of antiapoptotic proteins bcl-2 in BPH-1 cells. Western blot of pro-apoptotic proteins Bax and p53 and anti-apoptotic protein Bcl-2 were performed from whole cell lysates of BPH-1 cells treated with XN and XAL for 48 h. Blots are representative of three individual experiments.

### BRIEF SUMMARY OF THE INVENTION

It has been discovered that, surprisingly, xanthohumol and xanthohumol analogs may be used to treat BPH or prostate cancer.

In one aspect, a method of treating or preventing prostate cancer or BPH in a subject in need of such treatment is provided. The method includes administering to the subject an effective amount of xanthohumol or xanthohumol analog (e.g. a xanthohumol metabolite).

In another aspect, a method of decreasing cell viability in a prostate cancer cell or a BPH cell is provided. The method includes contacting the cell with xanthohumol or xanthohumol analog (e.g. metabolite).

In another aspect, a method of increasing apoptosis in a prostate cancer cell or a BPH cell is provided. The method includes administering to the cell xanthohumol or xanthohumol analog (e.g. metabolite).

In another aspect, a method of treating or preventing a symptom of benign prostatic hyperplasia (BPH) in a subject in need of such treatment is provided. The method includes administering to the subject an effective amount of xanthohumol or xanthohumol analog (e.g. metabolite).

### DETAILED DESCRIPTION OF THE INVENTION

### I. Definitions

The term “pharmaceutically acceptable salts” is meant to include salts of the active compounds which are prepared with relatively nontoxic acids or bases, depending on the particular substituents found on the compounds described herein. When the active compounds contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain compounds contain both basic and acidic functionalities that allow the compounds to be converted into either base or acid addition salts.

The neutral forms of the xanthohumol or xanthohumol analogs may be regenerated by contacting the salt with a base or acid and isolating the parent compound in the conventional manner. The parent form of the compound differs from the various salt forms in certain physical properties, such as solubility in polar solvents.

In addition to salt forms, the xanthohumol or xanthohumol analogs are provided which are in a prodrug form. Prodrugs of the xanthohumol or xanthohumol analog compounds described herein are those compounds that readily undergo chemical changes under physiological. Additionally, prodrugs can be converted to the xanthohumol or xanthohumol analog compounds by chemical or biochemical methods in an ex vivo environment. For example, prodrugs can be slowly converted to the xanthohumol or xanthohumol analogs when placed in a transdermal patch reservoir with a suitable enzyme or chemical reagent.

Certain xanthohumol or xanthohumol analogs can exist in unsolvated forms as well as solvated forms, including hydrated forms. In general, the solvated forms are equivalent to unsolvated forms. Certain the xanthohumol or xanthohumol analogs may exist in multiple crystalline or amorphous forms. In general, all physical forms are equivalent for the uses contemplated by the methods provided herein.

Certain xanthohumol or xanthohumol analogs may possess asymmetric carbon atoms (optical centers) or double bonds; the racemates, diastereomers, geometric isomers and individual isomers.

The xanthohumol or xanthohumol analogs may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C).

The term “treating” refers to any indicia of success in the treatment or amelioration of prostate cancer, including any objective or subjective parameter such as abatement; remission; diminishing of symptoms or making the prostate cancer, pathology or condition more tolerable to the patient; slowing in the rate of degeneration or decline; or making the final point of degeneration less debilitating. The treatment or amelioration of symptoms can be based on objective or subjective parameters. For example, the certain methods presented herein successfully treat prostate cancer by decreasing the incidence of cancer and or causing remission of cancer.

The term “preventing” as used herein refers to either preventing the onset of prostate cancer, or preventing the onset of a preclinically evident stage of prostate cancer in individuals at risk. Also intended to be encompassed by this definition is the prevention of initiation for malignant cells, and the arrest or reversal of the progression of premalignant cells to malignant cells. “Preventing” also includes the prevention of growth or spreading of the prostate cancer. This includes prophylactic treatment of those at risk of developing a prostate cancer.

Administration of an agent “in combination with” includes parallel administration (administration of both the agents to the patient over a period-of time), co-administration (in which the agents are administered at approximately the same time, e.g., within about a few minutes to a few hours of one another), and co-formulation (in which the agents are combined or compounded into a single dosage form suitable for oral or parenteral administration).

An “effective amount” is an amount sufficient to contribute to the treatment or prevention of a stated disease state or to contribute to a decrease in cell viability in a stated cell type. Where the effective amount refers to the treatment of a stated disease state, the amount may also be referred to as a “therapeutically effective amount.”

As used herein, “reduction” of a symptom or symptoms (and grammatical equivalents of this phrase) means decreasing of the severity or frequency of the symptom(s), or elimination of the symptom(s).

As used herein, “administering” or “administration of” a drug to a subject (and grammatical equivalents of this phrase) includes both direct administration, including self-administration, and indirect administration, including the act of prescribing a drug. For example, as used herein, a physician who instructs a patient to self-administer a drug and/or provides a patient with a prescription for a drug is administering the drug to the patient.

As used herein, a “manifestation” of BPH refers to a symptom, sign, anatomical state (e.g., prostate size), physiological state (e.g., PSA level), or report (e.g., AUASI score) characteristic of a subject with BPH.

As used herein, a “prophylactically effective amount” of a drug is an amount of a drug that, when administered to a subject, will have the intended prophylactic effect, e.g., preventing or delaying the onset (or reoccurrence) of disease or symptoms, or reducing the likelihood of the onset (or reoccurrence) of disease or symptoms. The full prophylactic effect does not necessarily occur by administration of one dose, and may occur only after administration of a series of doses. Thus, a prophylactically effective amount may be administered in one or more administrations.

The term “cancer” as used herein refers broadly to neoplastic, pre-malignant, and proliferative disease, including specifically benign, premalignant, or malignant neoplasms in individuals with or without any prior history or diagnosis of neoplastic, pre-malignant, or proliferative disease.

The term “prostate cancer” as used herein refers broadly to epithelial cancers, epitheliomas, carcinomas, sarcomas, or other malignant tumors or cancer of glandular origin in the prostate.

“Patient” refers to a mammalian subject (e.g. human). The term “subject” as used herein refers to any human or mammal. The subject may be a human subject. In some embodiments, the subject is a male human subject. For methods of prevention, the subject is typically any human or animal subject at risk for developing an epithelial cell-derived prostate cancer. The subject may be at risk due to exposure to carcinogenic agents, being genetically predisposed to have a prostate cancer, BPH, and the like.

### II. Methods of Treatment

In one aspect, a method of treating or preventing prostate cancer in a subject in need of such treatment is provided. The method includes administering to the subject an effective amount of xanthohumol or xanthohumol analog (e.g. a xanthohumol metabolite). The methods disclosed herein are effective in treating or preventing any appropriate stage of prostate cancer, including for example, early stage prostate cancer such as pre-malignant stages. Thus, in some embodiments, the prostate cancer is early stage prostate cancer.

In another aspect, a method of treating or preventing benign prostatic hyperplasia in a subject in need of such treatment is provided. The method includes administering to the subject an effective amount of xanthohumol or xanthohumol analog (e.g. metabolite).

In another aspect, a method of treating or preventing a symptom of benign prostatic hyperplasia (BPH) in a subject in need of such treatment is provided. The method includes administering to the subject an effective amount of xanthohumol or xanthohumol analog (e.g. metabolite). In some embodiments, the symptom is a male urinary symptom, such as incomplete bladder emptying, high urination frequency, urination intermittency, high urination urgency, weak urination stream, or straining to commence urination.

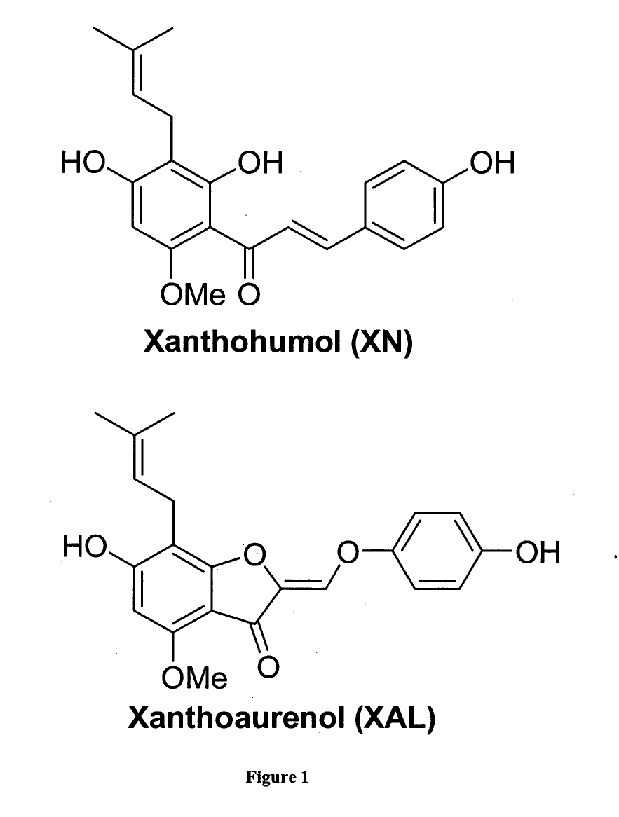

In some embodiments, xanthohumol is administered to the subject. See FIG. 1. In other embodiments, compounds related to xanthohumol are useful for the treatment and prevention of BPH and/or prostate cancer. Useful compounds are metabolites of, compounds generally structurally similar to, bioisosteres of, and/or are pharmacophores of xanthohumol. Xanthohumol analogs have biological activity similar to those of xanthohumol. Such compounds can be referred as “bioactive xanthohumol analogs,” “xanthohumol analogs,” or, in some cases, simply, “analogs.” In addition, xanthohumol, xanthohumol analogs, and pharmaceutically acceptable salts thereof are used in the methods of the present invention.

In some embodiments, the xanthohumol analog used in the treatment methods of the invention is a xanthohumol metabolite. A xanthohumol metabolite refers to a compound produced by metabolism of xanthohumol, such as xanthoaurenol. In some embodiments, the xanthohumol metabolite is a compound produced by mammalian metabolism (e.g. human metabolism) of xanthohumol. The xanthohumol metabolite may be xanthoaurenol. See FIG. 1.

In addition, xanthohumol analogs that may be used in the treatment methods of the invention include bioisosteres and pharmacophores of xanthohumol and analogs described herein. Bioisosterism is a well-known tool for predicting the biological activity of compounds, based upon the premise that compounds with similar size, shape, and electron density can have similar biological activity. To form a bioisostere of a given molecule, one replaces one or more atoms or groups with known bioisosteric replacements for that atom or group. Known bioisosteric replacements include, for example, the interchangeability of —F, —OH, —NH₂, Cl, and —CH₃; the interchangeability of —Br and -iC₃H₇; the interchangeability of —I and -tC₄H₉; the interchangeability of —O—, —S—, —NH—, —CH₂, and —Se—; the interchangeability of —N═, —CH═, and —P═ (in cyclic or noncyclic moieties); the interchangeability of phenyl and pyridyl groups; the interchangeability of —C═C— and —S— (for example, benzene and thiophene); the interchangeability of an aromatic nitrogen for an unsaturated carbon; and the interchangeability of —CO—, —SO—, and —SO₂—. These examples are not limiting on the range of bioisosteric equivalents and one of skill in the art will be able to identify other bioisosteric replacements known in the art.

Xanthohumol analogs that may be used in the methods of the invention can additionally be any pharmacophore of xanthohumol. Often a reasonable quantitative prediction of the binding ability of a known molecule can be made based on the spatial arrangement of a small number of atoms or functional groups in the molecule. Such an arrangement is called a pharmacophore, and once the pharmacophore or pharmacophores in a molecule have been identified, this information can be used to identify other molecules containing the same or similar pharmacophores. Such methods are well known to persons of ordinary skill in the art of medicinal chemistry, and as the structural information described in this application identifies the pharmacophore of xanthohumol and the xanthohumol analogs relevant to treatments of BPH and prostate cancer, those of skill in the art can identify other xanthohumol analogs that comprise the pharmacophore and so are useful in treating BPH and/or prostate cancer. An example of programs available to perform pharmacophore-related searches is the program 3D Pharmacophore search from the Chemical Computing Group.

In some embodiments, xanthohumol or an analog thereof is administered to a subject with a serum PSA level greater than 2 ng/ml. PSA is secreted only by the epithelial cells of the prostate. For men with BPH, higher PSA levels suggest a relatively higher ratio of epithelial cell proliferation to stromal cell proliferation than in men with lower PSA levels. A number of diagnostic methods are suitable for use in determining patients who should respond favorably to treatment with xanthohumol or an analog. Thus, xanthohumol treatment can provide a therapeutic benefit to subjects with PSA levels greater than 2 ng/ml. Accordingly, subjects predicted to benefit significantly from treatment in accordance with the invention can be selected in a population of men with BPH by identifying subjects with a serum PSA value greater than 2 ng/ml. In one embodiment of the invention, the subject has a PSA level greater than about 4 ng/ml. In some embodiments, where the subject is being evaluated for BPH because of higher PSA levels, the subject selected for therapy with xanthohumol or an analog has a PSA level less than about 10 ng/ml.

Any references in the methods provided herein to administration of xanthohumol also apply to administration of a xanthohumol analog.

In another aspect, the invention provides a method entailing (a) advertising the use of xanthohumol, or a xanthohumol analog, for treatment of BPH or prostate cancer, and (b) selling xanthohumol or a xanthohumol analog to individuals for use for treatment of BPH or prostate cancer. In one embodiment, the advertising makes reference to a trademark that identifies a xanthohumol product and the xanthohumol sold in step (b) is identified by the same trademark. It will be appreciated that the individuals to whom xanthohumol is sold include corporate persons (corporations) and the like and “selling BPH to individuals for use for treatment of BPH” includes selling to, for example, a medical facility for distribution to patients for treatment of BPH.

A. Treatment of BPH and BPH Symptoms

Administration of xanthohumol (or analog thereof) to a subject diagnosed with, or exhibiting symptoms of, BPH provides benefits such as reduction of severity or frequency of one or more symptoms, reduction in prostate size or rate of enlargement, improvement in perceived quality of life, and reversion of other manifestations of BPH toward a more normal state. Further, administration of xanthohumol or xanthohumol analogs to a subject in need of prophylaxis for BPH provides benefits such as a reduction in likelihood that BPH will appear, reappear or progress in the subject.

In another embodiment, administration of xanthohumol or its analogs to a human subject as described herein can be efficacious in the treatment of acute urinary retention. These and other aspects of the invention are discussed in greater detail below.

Desired clinical results of treatment for BPH include, but are not limited to, alleviation or amelioration of one or more symptoms of BPH, a reduction in prostate, a reduction in AUASI or IPSS scores compared to base line measurements prior to commencement of therapy, AUASI or IPSS scores less than 8, a reduction in serum PSA by at least about 20%, such as by at least about 40%, a serum PSA less than 4, such as less than 2, improvement in urodynamic parameters, and other desired results that will be recognized by a treating physician as indicative of a reduction in severity of BPH in a subject. An assessment of the response to treatment can be made at any time following the first administration of the drug. For example, an assessment is made about 30 days, about 60 days, or about 90 days after beginning treatment. Alternatively, assessment can be made about 6, 12, 18, 24 or more months after beginning treatment. Alternatively, an assessment can be made less than about 30 days, about 30 days, about 60 days, or about 90 days after a course of treatment ends.

As used herein, “a symptom associated with BPH” or “a symptom of BPH” includes male urinary symptoms such as one or more of the following symptoms: (1) high urinary urgency; (2) terminal dribbling of urine; (3) high urination frequency (also referred to herein as frequent urination); (4) nocturia; (5) a weak/slow stream of urine; (6) incomplete bladder emptying or a sense of incomplete emptying; (7) urination intermittency; (8) straining to commence urination; (9) dysuria; (10) hematuria; (11) acute urinary retention; (12) urinary tract infection; and (13) incontinence. Administration of xanthohumol or a xanthohumol analog according to the methods of the invention typically results in a reduction in severity, or elimination, of one or more of these symptoms; usually results in either a reduction in severity of, or elimination of, all of these symptoms; and often results in elimination of all of these symptoms.

In some embodiments, xanthohumol or a xanthohumol analog is administered to reduce prostate size in a human subject in need of such reduction. As used herein, “a subject in need of reduction of prostate size” is a subject (e.g. a man) having an enlarged prostate gland as determined by (1) imaging (e.g., ultrasonography, magnetic resonance imaging) or (2) one or more signs or symptoms resulting directly or indirectly from compression of the urethra by the prostate (e.g., including the symptoms of BPH discussed herein). A reduction in serum PSA (prostate specific antigen) is also a useful proxy for reduction of prostate volume. Although varying among individuals, enlarged prostates often exceed 30 grams, 40 grams, or 50 grams in size. The degree of reduction of prostate size will vary from subject to subject due to a number of factors, including the degree of enlargement at the time of onset of therapy, but will typically be a reduction of at least about 10% volume, more often at least about 25%, sometimes at least about 40%, sometimes at least about 50%, and sometimes an even greater than 50% reduction in prostate size is observed. This reduction can be determined by imaging or other methods. Serum PSA can also in some instances serve as a useful proxy for prostate volume.

In certain embodiments, xanthohumol or a xanthohumol analog is administered to a subject who would benefit from prophylaxis of BPH. In one example, “a subject who would benefit from prophylaxis of BPH” is a subject (e.g. a man) previously treated for BPH by surgery, transurethral microwave thermotherapy, transurethral needle ablation, transurethral electrovaporization, laser therapy, balloon dilatation, prostatic urethral stent, drug therapy, or other therapy and not currently diagnosed with or exhibiting symptoms of BPH. In another example, a subject who would benefit from prophylaxis of BPH is a man at increased risk for developing BPH due to age (e.g., men older than 40, older than 50, older than 60, or older than 70 years of age). In another example, a subject who would benefit from prophylaxis of BPH is a man who is asymptomatic, or has symptoms sufficiently mild so that no clear diagnosis of BPH can be made, but who has an elevated serum PSA level (e.g., PSA>2 ng/ml or, in some cases, >4 ng/ml).

Thus, in some cases, the subject to whom xanthohumol is administered in accordance with the methods of the invention is a man who has previously been treated for BPH, while in other cases the subject is a man who has not previously been treated for BPH.

In another embodiment, the invention provides a method of treating acute urinary retention in a subject by administering xanthohumol or a xanthohumol analog to the subject. Because acute urinary retention can be a symptom of BPH, this embodiment of the invention is applicable to any subject who suffers from acute urinary retention but has not been diagnosed as having BPH when xanthohumol or a xanthohumol analog is first administered.

### III. Cellular Methods

In another aspect, a method of decreasing cell viability in a prostate cancer cell is provided. The method includes contacting the cell with xanthohumol or xanthohumol analog (e.g. metabolite). The amount of xanthohumol or xanthohumol metabolite is an effective amount to contribute to decreasing the cell viability of the prostate cancer cell.

Any appropriate prostate cancer cell may be subjected to the methods of decreasing cell viability. In some embodiments, the prostate cancer cell is a malignant prostate cancer cell. In other embodiments, the prostate cancer cell is a malignant prostate cancer epithelial cell. In other embodiments, the prostate cancer cell is a malignant androgen-independent epithelial cell.

In another aspect, a method of decreasing cell viability in a benign prostatic hyperplasia cell is provided. The method includes, contacting the cell with an effective amount of xanthohumol or xanthohumol analog (e.g. metabolite).

Any benign prostatic hyperplasia cell may be subjected to the methods of decreasing cell viability. In some embodiments, the benign prostatic hyperplasia cell is a human benign prostatic hyperplasia cell. In other embodiments, the benign prostatic hyperplasia cell is a human benign prostatic hyperplasia epithelial cell.

The decrease in cell viability is relative to the cell viability observed in a population of the cell in the absence of the xanthohumol or xanthohumol analog (e.g. more of the cells are viable as compared to non-exposure to (contact with) the xanthohumol or xanthohumol analog). Cell viability, as used herein, refers to the ability of a cell to live, grow, and/or develop. Therefore, an effective amount of the xanthohumol or xanthohumol analog is introduced into the cell to decrease the number of viable cells. In some embodiments, the methods of decreasing cell viability results in reducing the number of living cells.

In another aspect, a method of increasing apoptosis in a cell is provided. The method includes administering to the cell xanthohumol or its analog (e.g. metabolite). In some embodiments, the cell is a prostate cancer cell or a prostatic benign hyperplasia cell. Applicable prostate cancer cells and prostatic benign hyperplasia cells are discussed above.

The increase in apoptosis is relative to the incidence of apoptosis observed in a population of the cell in the absence of the xanthohumol or xanthohumol analog (e.g. more of the cells are induced into the death process as compared to non-exposure to (contact with) the xanthohumol or xanthohumol analog). Thus, an effective amount of the xanthohumol or xanthohumol analog is introduced into the cell to result in an increase in apoptosis. Apoptosis is generally considered to be a form of cell death in which a controlled sequence of events (or program) leads to the elimination of the cell. Increasing apoptosis also includes the inhibition of cell division.

The term “contacting” in reference to the methods of contacting cells with xanthohumol or xanthohumol derivatives means that the cell is exposed to xanthohumol or xanthohumol derivative under conditions in which the xanthohumol or xanthohumol derivative is introduced into the cell.

Any references in the methods provided herein to contacting a cell with xanthohumol also applies to contacting the cell with a xanthohumol analog.

### IV. Additional Agents for Use in Prostate Cancer Treatments

In some embodiments, the methods include the administration to a subject, or the contacting of a cell, with an additional agent such as an antineoplastic (i.e. anticancer agents), radioprotective agent, a growth inhibiting agent, and/or nutrient.

In some embodiments, the antineoplastic agent is an antimetabolite agent, antibiotic-type agent, alkylating agent, hormonal agent, immunological agent, interferon-type agent, metallomatrix protease, superoxide dismutase mimic or αᵥβ₃ inhibitor. Examples of suitable antimetabolite antineoplastic agents include 5-FU-fibrinogen, acanthifolic acid, aminothiadiazole, brequinar sodium, carmofur, Ciba-Geigy CGP-30694, cyclopentyl cytosine, cytarabine phosphate stearate, cytarabine conjugates, Lilly DATHF, Merrel Dow DDFC, dezaguanine, dideoxycytidine, dideoxyguanosine, didox, Yoshitomi DMDC, doxifluridine, Wellcome EHNA, Merck & Co. EX-015, fazarabine, floxuridine, fludarabine phosphate, 5-fluorouracil, N-(2′-furanidyl)-5-fluorouracil, Daiichi Seiyaku OF-152, isopropyl pyrrolizine, Lilly LY-188011, Lilly LY-264618, methobenzaprim, methotrexate, Wellcome MZPES, norspermidine, NCI NSC-127716, NCI NSC-264880, NCI NSC-39661, NCI NSC-612567, Warner-Lambert PALA, pentostatin, piritrexim, plicamycin, Asahi Chemical PL-AC, Takeda TAC-788, thioguanine, tiazofurin, Erbamont TIF, trimetrexate, tyrosine kinase inhibitors, tyrosine protein kinase inhibitors, Taiho UFT, and uricytin.

Examples of suitable alkylating-type antineoplastic agents include Shionogi 254-S, aldo-phosphamide analogues, altretamine, anaxirone, Boehringer Mannheim BBR-2207, bestrabucil, budotitane, Wakunaga CA-102, carboplatin, carmustine, Chinoin-139, Chinoin-153, chlorambucil, cisplatin, cyclophosphamide, American Cyanamid CL-286558, Sanofi CY-233, cyplatate, Degussa D-19-384, Sumimoto DACHP(Myr)2, diphenylspiromustine, diplatinum cytostatic, Erba distamycin derivatives, Chugai DWA-2114R, ITI E09, elmustine, Erbamont FCE-24517, estramustine phosphate sodium, fotemustine, Unimed G-6-M, Chinoin GYKI-17230, hepsul-fam, ifosfamide, iproplatin, lomustine, mafosfamide, mitolactol, Nippon Kayaku NK-121, NCI NSC-264395, NCI NSC-342215, oxaliplatin, Upjohn PCNU, prednimustine, Proter PTT-119, ranimustine, semustine, SmithKline SK&F-101772, Yakult Honsha SN-22, spiromustine, Tanabe Seiyaku TA-077, tauromustine, temozolomide, teroxirone, tetraplatin, and trimelamol.

Examples of suitable antibiotic-type antineoplastic agents include Taiho 4181-A, aclarubicin, actinomycin D, actinoplanone, Erbamont ADR-456, aeroplysinin derivative, Ajinomoto AN-201-II, Ajinomoto AN-3, Nippon Soda anisomycins, anthracycline, azino-mycin-A, bisucaberin, Bristol-Myers BL-6859, Bristol-Myers BMY-25067, Bristol-Myers BMY-25551, Bristol-Myers BMY-26605, Bristol-Myers BMY-27557, Bristol-Myers BMY-28438, bleomycin sulfate, bryostatin-1, Taiho C-1027, calichemycin, chromoximycin, dactinomycin, daunorubicin, Kyowa Hakko DC-102, Kyowa Hakko DC-79, Kyowa Hakko DC-88A, Kyowa Hakko DC89-A1, Kyowa Hakko DC92-B, ditrisarubicin B, Shionogi DOB-41, doxorubicin, doxorubicin-fibrinogen, elsamicin-A, epirubicin, erbstatin, esorubicin, esperamicin-A1, esperamicin-A1b, Erbamont FCE-21954, Fujisawa FK-973, fostriecin, Fujisawa FR-900482, glidobactin, gregatin-A, grincamycin, herbimycin, idarubicin, illudins, kazusamycin, kesarirhodins, Kyowa Hakko KM-5539, Kirin Brewery KRN-8602, Kyowa Hakko KT-5432, Kyowa Hakko KT-5594, Kyowa Hakko KT-6149, American Cyanamid LL-D49194, Meiji Seika ME 2303, menogaril, mitomycin, mitoxantrone, SmithKline M-TAG, neoenactin, Nippon Kayaku NK-313, Nippon Kayaku NKT-01, SRI International NSC-357704, oxalysine, oxaunomycin, peplomycin, pilatin, pirarubicin, porothramycin, pyrindamycin A, Tobishi RA-I, rapamycin, rhizoxin, rodorubicin, sibanomicin, siwenmycin, Sumitomo SM-5887, Snow Brand SN-706, Snow Brand SN-07, sorangicin-A, sparsomycin, SS Pharmaceutical SS-21020, SS Pharmaceutical SS-7313B, SS Pharmaceutical SS-9816B, steffimycin B, Taiho 4181-2, talisomycin, Takeda TAN-868A, terpentecin, thrazine, tricrozarin A, Upjohn U-73975, Kyowa Hakko UCN-10028A, Fujisawa WF-3405, Yoshitomi Y-25024, and zorubicin.

Other examples of suitable antnineoplastic agents include alpha-carotene, alpha-difluoromethyl-arginine, acitretin, Biotec AD-5, Kyorin AHC-52, alstonine, amonafide, amphethinile, amsacrine, Angiostat, ankinomycin, anti-neoplaston A10, antineoplaston A2, antineoplaston A3, antineoplaston A5, antineoplaston AS2-1, Henkel APD, aphidicolin glycinate, asparaginase, Avarol, baccharin, batracylin, benfluoron, benzotript, Ipsen-Beaufour BIM-23015, bisantrene, Bristo-Myers BMY-40481, Vestar boron-10, bromofosfamide, Wellcome BW-502, Wellcome BW-773, caracemide, carmethizole hydrochloride, Ajinomoto CDAF, chlorsulfaquinoxalone, Chemes CHX-2053, Chemex CHX-100, Warner-Lambert CI-921, Warner-Lambert CI-937, Warner-Lambert CI-941, Warner-Lambert CI-958, clanfenur, claviridenone, ICN compound 1259, ICN compound 4711, Contracan, Yakult Honsha CPT-11, crisnatol, curaderm, cytochalasin B, cytarabine, cytocytin, Merz D-609, DABIS maleate, dacarbazine, datelliptinium, didemnin-B, dihaematoporphyrin ether, dihydrolenperone, dinaline, distamycin, Toyo Pharmar DM-341, Toyo Pharmar DM-75, Daiichi Seiyaku DN-9693, elliprabin, elliptinium acetate, Tsumura EPMTC, ergotamine, etoposide, etretinate, fenretinide, Fujisawa FR-57704, gallium nitrate, genkwadaphnin, Chugai GLA-43, Glaxo GR-63178, grifolan NMF-5N, hexadecylphosphocholine, Green Cross HO-221, homoharringtonine, hydroxyurea, BTG ICRF-187, ilmofosine, isoglutamine, isotretinoin, Otsuka JI-36, Ramot K-477, Otsuak K-76COONa, Kureha Chemical K-AM, MECT Corp KI-8110, American Cyanamid L-623, leukoregulin, xanthohumol, Lundbeck LU-23-112, Lilly LY-186641, NCI (US) MAP, marycin, Merrel Dow MDL-27048, Medco MEDR-340, merbarone, merocyanine derivatives, methylanilinoacridine, Molecular Genetics MGI-136, minactivin, mitonafide, mitoquidone, mopidamol, motretinide, Zenyaku Kogyo MST-16, N-(retinoyl)amino acids, Nisshin Flour Milling N-021, N-acylated-dehydroalanines, nafazatrom, Taisho NCU-190, nocodazole derivative, Normosang, NCI NSC-145813, NCI NSC-361456, NCI NSC-604782, NCI NSC-95580, octreotide, Ono ONO-112, oquizanocine, Akzo Org-10172, pancratistatin, pazelliptine, Warner-Lambert PD-111707, Warner-Lambert PD-115934, Warner-Lambert PD-131141, Pierre Fabre PE-1001, ICRT peptide D, piroxantrone, polyhaematoporphyrin, polypreic acid, Efamol porphyrin, probimane, procarbazine, proglumide, Invitron protease nexin I, Tobishi RA-700, razoxane, Sapporo Breweries RBS, restrictin-P, retelliptine, retinoic acid, Rhone-Poulenc RP-49532, Rhone-Poulenc RP-56976, SmithKline SK&F-104864, Sumitomo SM-108, Kuraray SMANCS, SeaPharm SP-10094, spatol, spirocyclopropane derivatives, spirogermanium, Unimed, SS Pharmaceutical SS-554, strypoldinone, Stypoldione, Suntory SUN 0237, Suntory SUN 2071, superoxide dismutase, Toyama T-506, Toyama T-680, taxol, Teijin TEI-0303, teniposide, thaliblastine, Eastman Kodak TJB-29, tocotrienol, Topostin, Teijin TT-82, Kyowa Hakko UCN-01, Kyowa Hakko UCN-1028, ukrain, Eastman Kodak USB-006, vinblastine sulfate, vincristine, vindesine, vinestramide, vinorelbine, vintriptol, vinzolidine, withanolides, and Yamanouchi YM-534.

In some embodiments, the xanthohumol or xanthohumol analog is used in combination with radiotherapy to treat prostate cancer. Thus, in some embodiments, the xanthohumol or xanthohumol analog is used in combination with radiotherapy and a radioprotective agent such as AD-5, adchnon, amifostine analogues, detox, dimesna, 1-102, MM-159, N-acylated-dehydroalanines, TGF-Genentech, tiprotimod, amifostine, WR-151327, FUT-187, ketoprofen transdermal, nabumetone, superoxide dismutase (Chiron), and superoxide dismutase Enzon.

In some embodiments, the additional agents are administered at a lower dosage (amount) or less frequently (e.g., alternate days rather than daily) than the “standard” dosage (the dosage that would be indicated for the subject in the absence of xanthohumol administration) in combination with xanthohumol or a xanthohumol analog. In some embodiments, the additional agents are administered at a lower dosage (amount) or less frequently (e.g., alternate days rather than daily) than the “standard” dosage (the dosage that would be indicated for the subject in the absence of xanthohumol administration) in combination with xanthohumol or a xanthohumol analog. In addition, xanthohumol or a xanthohumol analog may be administered in combination with, or prior to, procedures for treatment of prostate cancer including surgery, radiotherapy, or other non-drug therapies.

### V. Additional Agents for Use in BPH Treatments

Xanthohumol and xanthohumol analogs can be administered to a BPH patient in combination with other agents or procedures intended to treat BPH, ameliorate symptoms of BPH, potentiate the effects of the xanthohumol or xanthohumol analog, or provide other therapeutic benefit. Exemplary agents for administration in combination with xanthohumol or xanthohumol analogs in the treatment of BPH or symptoms thereof include, but are not limited to, zinc (e.g., zinc chloride, zinc gluconate, zinc sulfate, zinc acetate, zinc aspartate, zinc citrate, zinc glycerate, zinc oxide, zinc picolinate, etc.), alpha-blockers, 5-alpha-reductase inhibitors, and plant extracts. Other agents for administration in combination with xanthohumol or xanthohumol analogs include other metabolic inhibitors, including but not limited to other hexokinase inhibitors and other inhibitors of glycolysis, including but not limited to 2-deoxy-D-glucose and an inhibitor, direct or indirect, of HIF-1α.

Exemplary alpha-blockers include doxazosin (Cardura), terazosin (Hytrin), tamsulosin (Flomax), alfuzosin (Xatral), and prazosin (Hypovase). An exemplary 5-alpha-reductase inhibitor is finasteride (Proscar). Glycolytic inhibitors, such as 2-deoxy-D-glucose and compounds that inhibit glucose transport, mitochondrial function inhibitors, mitochondrial poisons, and hexokinase inhibitors such as 3-bromopyruvate and its analogs can also be used in combination with xanthohumol or a xanthohumol analog to treat BPH. See WO 01/82926 published 8 Nov. 2001; U.S. Pat. Nos. 6,670,330; 6,218,435; 5,824,665; 5,652,273; and 5,643,883; U.S. patent application publication Nos. 2003/0072814; 2002/0077300; and 2002/0035071; and U.S. patent application Ser. No. 10/754,239, each of which are incorporated by reference for all purposes. Examples of useful plant materials include Saw Palmetto (Serenoa repens) or an extract thereof, or Pygeum Africanum or an extract thereof.

In some embodiments, the additional agents are administered at a lower dosage (amount) or less frequently (e.g., alternate days rather than daily) than the “standard” dosage (the dosage that would be indicated for the subject in the absence of xanthohumol administration) in combination with xanthohumol or a xanthohumol analog. In addition, xanthohumol or a xanthohumol analog may be administered in combination with, or prior to, procedures for treatment of BPH including surgery (transurethral resection of the prostate; transurethral incision of the prostate; or open prostatectomy), laser therapy, transurethral microwave thermotherapy, balloon dilatation, placement of a prostatic urethral stent, transurethral needle ablation, transurethral electrovaporization of the prostate, or other non-drug therapies.

### VI. Pharmaceutical Compositions

Xanthohumol and xanthohumol analogs may be administered to the subject by means of a pharmaceutically acceptable carrier. Such carriers generally will be in either solid or liquid form. Solid form pharmaceutical preparations which may be prepared according to the present inventive subject matter include powders, tablets, dispersible granules, capsules, and cachets. In general, solid form preparations will comprise from about 5% to about 90% by weight of the active agent.

A solid carrier can be one or more substances which may also act as diluents, flavoring agents, solubilizers, lubricants, suspending agents, binders or tablet disintegrating agents; it can also be an encapsulating material. In powders, the carrier is a finely divided solid which is in admixture with the viscous active compound. In tablets, the active compound is mixed with a carrier having the necessary binding properties in suitable proportions and compacted to the shape and size desired. Suitable solid carriers include magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term preparation is intended to include the formulation of the active compound with encapsulating materials as a carrier which may provide a capsule in which the active component (with or without other carriers) is surrounded by carrier, which is thus in association with it. Similarly, cachets are included. Tablets, powders, cachets, and capsules can be used as solid dosage forms suitable for oral administration. If desired for reasons of convenience or patient acceptance, pharmaceutical tablets prepared according to the inventive subject matter may be provided in chewable form.

Suitable carriers also include solid form preparations which are intended to be converted, shortly before use, to liquid form preparations for either oral or parenteral administration. Such liquid forms include solutions, suspensions, and emulsions. These particular solid form preparations are most conveniently provided in unit dose form and as such are used to provide a single liquid dosage unit. Alternately, sufficient solid may be provided so that after conversion to liquid form, multiple individual liquid doses may be obtained by measuring predetermined volumes of the liquid form preparation as with a syringe, teaspoon, or other volumetric container. When multiple liquid doses are so prepared, it is preferred to maintain the unused portion of said liquid doses at low temperature (i.e., under refrigeration) in order to retard possible decomposition. The solid form preparations intended to be converted to liquid form may contain, in addition to the active material, flavorants, colorants, stabilizers, buffers, artificial and natural sweeteners, dispersants, thickeners, solubilizing agents, and the like. The liquid utilized for preparing useful liquid form preparations may be water, isotonic water, ethanol, glycerine, propylene glycol, and the like as well as mixtures thereof. Naturally, the liquid utilized will be chosen with regard to the route of administration. For example, liquid preparations containing large amounts of ethanol are not suitable for parenteral use.

The pharmaceutical preparation may also be in a unit dosage form. In such form, the preparation is subdivided into unit doses containing appropriate quantities of the active component. The unit dosage form can be a packaged preparation, the package containing discrete quantities of preparation, for example, packeted tablets, capsules, and powders in vials or ampoules. The unit dosage form can also be a capsule, cachet, or tablet itself or it can be the appropriate number of any of these in packaged form.

The pharmaceutical preparations of the inventive subject matter may include one or more preservatives well known in the art, such as benzoic acid, sorbic acid, methylparaben, propylparaben and ethylenediaminetetraacetic acid (EDTA). Preservatives are generally present in amounts up to about 1% and preferably from about 0.05 to about 0.5% by weight of the pharmaceutical composition.

Useful buffers for purposes of the inventive subject matter include citric acid-sodium citrate, phosphoric acid-sodium phosphate, and acetic acid-sodium acetate in amounts up to about 1% and preferably from about 0.05 to about 0.5% by weight of the pharmaceutical composition. Useful suspending agents or thickeners include cellulosics like methylcellulose, carageenans like alginic acid and its derivatives, xanthan gums, gelatin, acacia, and microcrystalline cellulose in amounts up to about 20% and preferably from about 1% to about 15% by weight of the pharmaceutical composition.

Sweeteners which may be employed include those sweeteners, both natural and artificial, well known in the art. Sweetening agents such as monosaccharides, disaccharides and polysaccharides such as xylose, ribose, glucose, mannose, galactose, fructose, dextrose, sucrose, maltose, partially hydrolyzed starch or corn syrup solids and sugar alcohols such as sorbitol, xylitol, mannitol and mixtures thereof may be utilized in amounts from about 10% to about 60% and preferably from about 20% to about 50% by weight of the pharmaceutical composition. Water soluble artificial sweeteners such as saccharin and saccharin salts such as sodium or calcium, cyclamate salts, acesulfame-K, aspartame and the like and mixtures thereof may be utilized in amounts from about 0.001% to about 5% by weight of the composition.

Flavorants which may be employed in the pharmaceutical products of the inventive subject matter include both natural and artificial flavors, and mints such as peppermint, menthol, vanilla, artificial vanilla, chocolate, artificial chocolate, cinnamon, various fruit flavors, both individually and mixed, in amounts from about 0.5% to about 5% by weight of the pharmaceutical composition.

Colorants useful in the present inventive subject matter include pigments which may be incorporated in amounts of up to about 6% by weight of the composition. A preferred pigment, titanium dioxide, may be incorporated in amounts up to about 1%. Also, the colorants may include other dyes suitable for food, drug and cosmetic applications, known as F.D.&C. dyes and the like. Such dyes are generally present in amounts up to about 0.25% and preferably from about 0.05% to about 0.2% by weight of the pharmaceutical composition. A full recitation of all F.D.&C. and D.&C. dyes and their corresponding chemical structures may be found in the Kirk-Othmer Encyclopedia of Chemical Technology, in Volume 5, at pages 857 884, which text is accordingly incorporated herein by reference.

Useful solubilizers include alcohol, propylene glycol, polyethylene glycol and the like and may be used to solubilize the flavors. Solubilizing agents are generally present in amounts up to about 10%; or from about 2% to about 5% by weight of the pharmaceutical composition. Lubricating agents which may be used when desired in the instant compositions include silicone oils or fluids such as substituted and unsubstituted polysiloxanes, e.g., dimethyl polysiloxane, also known as dimethicone.

Pharmaceutical compositions containing xanthohumol or xanthohumol analogs may be administered in combination with other compounds and compositions useful for treating prostate cancer or BPH, as described above.

Pharmaceutical formulations may be developed depending upon considerations such as the route of administration and desired dosage. See, for example, “Remington's Pharmaceutical Sciences”, 18th ed. (1990, Mack Publishing Co., Easton, Pa. 18042), pp. 1435 1712, which is hereby incorporated by reference in its entirety.

The compounds and compositions may be administered orally in the form of capsules, tablets, aqueous suspensions, or solutions. Tablets may contain carriers such as lactose and corn starch, and/or lubricating agents such as magnesium stearate. Capsules may contain diluents including lactose and dried corn starch. Aqueous suspensions may contain emulsifying and suspending agents combined with the active ingredient. The oral dosage forms may further contain sweetening, flavoring, coloring agents, or combinations thereof. Delivery in an enterically coated tablet, caplet, or capsule, to further enhance stability and provide release in the intestinal tract to improve absorption, is the best mode of administration currently contemplated.

### VII. Dosage

Dosage forms (i.e. pharmaceutical compositions or pharmaceutical formulations) suitable for internal administration contain from about 1.0 milligram to about 5000 milligrams of active ingredient per unit. In these pharmaceutical compositions, the active ingredient may be present in an amount of about 0.5 to about 95% by weight based on the total weight of the composition. Another convention for denoting the dosage form is in mg per meter squared (mg/m²) of body surface area (BSA). Typically, an adult will have approximately 1.75 m² of BSA. Based on the body weight of the patient, the dosage may be administered in one or more doses several times per day or per week. Multiple dosage units may be required to achieve a therapeutically effective amount. For example, if the dosage form is 1000 mg, and the patient weighs 40 kg, one tablet or capsule will provide a dose of 25 mg per kg for that patient. It will provide a dose of only 12.5 mg/kg for a 80 kg patient.

By way of general guidance, for humans a dosage of as little as about 1 milligrams (mg) per kilogram (kg) of body weight and up to about 10000 mg per kg of body weight is suitable as a therapeutically effective dose. In certain embodiments, from about 5 mg/kg to about 2500 mg/kg of body weight is used. Other embodiments include doses range between 25 mg/kg to about 1000 mg/kg of body weight. However, a dosage of between about 2 milligrams (mg) per kilogram (kg) of body weight to about 400 mg per kg of body weight is also suitable for treating some cancers.

The dosage schedule and amounts effective for this use, i.e., the “dosing regimen,” will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra).

Single or multiple administrations of xanthohumol or xanthohumol analog can be administered depending on the dosage and frequency as required and tolerated by the patient. The formulations should provide a sufficient quantity of active agent to effectively treat the disease state. Lower dosages can be used, particularly when the drug is administered to an anatomically secluded site in contrast to administration orally, into the blood stream, into a body cavity or into a lumen of an organ. Substantially higher dosages can be used in topical administration. Actual methods for preparing parenterally administrable xanthohumol or xanthohumol analog formulations are described in more detail in such publications as Remington's, supra. See also Nieman, In “Receptor Mediated Antisteroid Action,” Agarwal, et al., eds., De Gruyter, New York (1987).

In some embodiments, the xanthohumol or xanthohumol analog is present in the formulation at a concentration of at least 5%, 10%, 20%, 25%, 30%, 35%, 45%, 45%, or 50% by weight. In other embodiments the xanthohumol or xanthohumol analog is present in the formulation at a concentration from 1% to 80%, 5% to 50%, 10% to 35%, or 20% to 25%.

In other embodiments, at least 0.5 mg, 1 mg, 2 mg, 3 mg, 4 mg, 5 mg, 10 mg, 20 mg, 30 mg, 40 mg, 50 mg, 100 mg, 200 mg, 300 mg, 400 mg, 500 mg, or 1 g of xanthohumol or xanthohumol analog is present in the formulation. In other embodiments, 0.1 mg to 2 g, 0.5 mg to 1 g, 1 mg to 500 mg, 1 mg to 100 mg, 1 mg to 50 mg, or 1 mg to 10 mg of xanthohumol or xanthohumol analog is present in the formulation.

### VIII. Assays

Cytotoxic assays in vitro for xanthohumol analogs generally involve the use of established prostate cancer cell lines or BPH cells both of animal and, especially of human origin. These cell lines can be obtained from commercial sources such as the American Type Tissue Culture Laboratory in Bethesda, Md. and from tumor banks at research institutions (e.g. human benign prostate hyperplasia epithelial cells (BPH-1) and malignant androgen-independent prostate cancer epithelial cells (PC-3)). Exposures to xanthohumol analog compounds may be carried out under simulated physiological conditions of temperature, oxygen and nutrient availability in the laboratory. The endpoints for these in vitro assays can involve: 1) colony formation; 2) a simple quantitation of cell division over time; 3) the uptake of so called “vital” dyes which are excluded from cells with an intact cytoplasmic membrane; 4) the incorporation of radiolabeled nutrients into a proliferating (viable) cell; 5) metabolism of marker compounds (e.g. MTT assay); 6) counting of the number of adherent and/or detached cells; 7) NFκB Activity; and 8) externalization of phosphatidyl serine. Colony forming assays have been used both with established cell lines, as well as fresh tumor biopsies surgically removed from patients with cancer. In this type of assay, cells are typically grown in petri dishes on soft agar, and the number of colonies or groups of cells (>60μ in size) are counted either visually, or with an automated image analysis system. A comparison is then made to the untreated control cells allowed to develop colonies under identical conditions.

The effect of a compound on prostate function, and, in particular, on respiration, can be assessed in an animal model or human by monitoring prostate tissue metabolism following administration of the compound. Some xanthohumol analogs useful in the present invention will detectably reduce ATP, citrate, and/or lactate production by the prostate in animals (including humans, non-human primates and other mammals). ATP, citrate, and/or lactate levels can be monitored directly and/or indirectly in vivo using techniques of magnetic resonance spectroscopy (MRS) or other methods. See, for example, Narayan and Kurhanewicz, 1992; Kurhanewicz et al., 1991; Thomas et al., 1990, for MRS assays that can be applied for this purpose.

The effect of a compound on prostate size can be assessed following administration of the compound using standard methods (for example, ultrasonography or digital rectal examination, for humans, and ultrasonography and/or comparison of organ weight in animals). Assays can be conducted in humans or, more usually, in healthy non-human animals or in monkey, dog, rat, or other animal models of BPH or prostate cancer (see, Jeyaraj et al., 2000; Lee et al., 1998; Mariotti et al., 1982).

Clinical trials can be used to assess the therapeutic effects of xanthohumol analogs.

The activity of a xanthohumol analog of interest in any of the aforementioned assays can be compared with that of xanthohumol to provide guidance concerning dosage schedules for the compound, and other information. Generally, xanthohumol analogs with greater biological activity per mg than xanthohumol are of special interest.

The terms and expressions which have been employed herein are used as terms of description and not of limitation, and there is no intention in the use of such terms and expressions of excluding equivalents of the features shown and described, or portions thereof, it being recognized that various modifications are possible within the scope of the invention claimed. Moreover, any one or more features of any embodiment of the invention may be combined with any one or more other features of any other embodiment of the invention where appropriate, without departing from the scope of the invention. For example, the applicable features of the methods of treating are equally applicable to the methods of contacting cells. All publications, patents, and patent applications cited herein are hereby incorporated by reference in their entirety for all purposes.

### IX. Examples

The following example are meant to merely illustrate certain embodiments of the technology disclosed herein, and is not meant to limit the scope of the invention.

### Cell Culture

Human benign prostate hyperplasia epithelial cells (BPH-1) were obtained from Dr. Simon W. Hayward (Vanderbilt University, Nashville, Tenn.) and malignant androgen-independent prostate cancer epithelial cells (PC-3) were obtained from American Type Tissue Collection (Manassas, Va.). Cells were grown and maintained in RPMI 1640 with glutamine (Mediatech, Inc., Herndon, Va.) supplemented with 5% (BPH-1) or 10% (PC-3) fetal bovine serum (Hyclone, Logan, Utah) and 1% penicillin-streptomycin (Mediatech, Inc., Herndon, Va.). All cell types were maintained in 5% CO₂ at 37° C.

### Treatments

XN was isolated from hop cones as described previously (Stevens et al., J. Chromatogr. A 832 (1999) 97-107). Its oxidation product, xanthoaurenol (XAL), was prepared from xanthohumol (XN) by treatment with peroxynitrite. The structure of XAL was determined by mass spectrometry, NMR spectroscopy and X-ray diffraction analysis. Prenylflavonoids were dissolved in 100% methanol and were added to the culture medium at a final concentration of 2.5-200 μmol/L, depending on desired treatment. All treatments were done at approximately 50-70% confluency. As a control, methanol was added to culture medium equivalent to hop treatment. Final concentration of methanol did not exceed 0.1% (FIG. 1).

### Cellular Proliferation

Cells were seeded into 24-well plates (50,000 cells per well). After 24 h, cells were treated with either XN or XAL as described above. Cell viability was evaluated by MTT assay as described by Mossmann et al., J. Immunol. Methods 65 (1983) 55-63. Production of formazan product was detected in a Molecular Devices SpectraMax (Molecular Devices, Sunnyvale, Calif.) at 580 nm.

### Flow Cytometry

Apoptosis

At various times after treatment with XN or XAL, adherent and floating cells were collected for analysis. Apoptosis was assessed using a flow cytometry based nexin and multicaspase assay kits (Guava Technologies, Burlingame, Calif.). The nexin assay is based on measurement of externalization of phosphatidyl serine, a common characteristic of cells undergoing apoptosis. The multicaspase assay is based on measurement of caspase enzymes activated during apoptosis. Cells were trypsinized, washed in D-PBS and stained with Annexin V, sulforhodamine-valyl-alanyl-aspartyl-fluoromethyl-ketone (SR-VAD-FMK), and 7-amino-actinomycin D (7AAD) according to the manufactures instructions. Cell populations were quantified using Guava personal cytometer (Guava Technologies, Burlingame, Calif.).

Cell Cycle Analysis

At various time points after treatment with XN and XAL, adherent and floating cells were collected and fixed in 70% ethanol at one million cells per aliqout. Samples were centrifuged at 500×g, washed with PBS, and resuspended in cellular DNA staining solution containing 40 mg/mL propidium iodide (Sigma, St Louis, Mo.) and 100 μg/mL RNase (Sigma, St Louis, Mo.) in PBS. After 30-min incubation at room temperature, cell populations were quantified using Guava personal cytometer. Data analysis was performed using Multi Cycle software (Phoenix Flow Systems, San Diego, Calif.).

NFκB Activation

NFκB Activity was measured using Trans AM NFκB p65/p50 Transcription Factor Assay Kit (Active Motif, CA). Cytosolic and nuclear extracts were prepared from cells by using Nuclear Extraction Kit (Active Motif, Carlsbad, Calif.). Nuclear extract samples (10 μg) were added to a 96 well plate with immobilized oligonucleotide containing the NFκB consensus site (5′-GGGACTTTCC-3′). Sample wells were incubated with primary antibody specific for NFκB, followed by incubation with an HRP-conjugated secondary antibody, followed by quantification at OD 580 using a plate reader (Molecular Devices, Sunnyvale, Calif.).

Western Blots

Cells were lysed in RIPA buffer and whole cell lysate protein concentrations were determined using a detergent-compatible protein assay (BioRad, CA). Proteins (15-35 μg) were separated by SDS-PAGE under standard conditions and were transferred to a nitrocellulose membrane (Biorad, CA) at 300 mA for 2 h. Antibody dilutions were as follows: Bax (BD Pharmingen, 1:500 dilution), Bcl-2 (Santa Cruz Biotechnology, 1:1000 dilution), p53 (Santa Cruz Biotechnology, 1:1000 dilution), NFκB p65 (Santa Cruz Biotechnology, 1:1000 dilution), actin (Sigma, 1:5000 dilution). Actin was used to confirm equal protein loading between samples. Protein was detected using chemiluminescence reagents (Pierce, Rockford, Ill.) with image analysis on Alpha Innotech photodocumentation system (Alpha Innotech, Hayward, Calif.).

Statistics

One-way analysis of variance (ANOVA) was performed to assess the differences between groups. Differences in means among treatments were tested by Dunnett's test, and the level of significance was designated as follows: *P<0.05, **P<0.01, ***P<0.001.

### Results

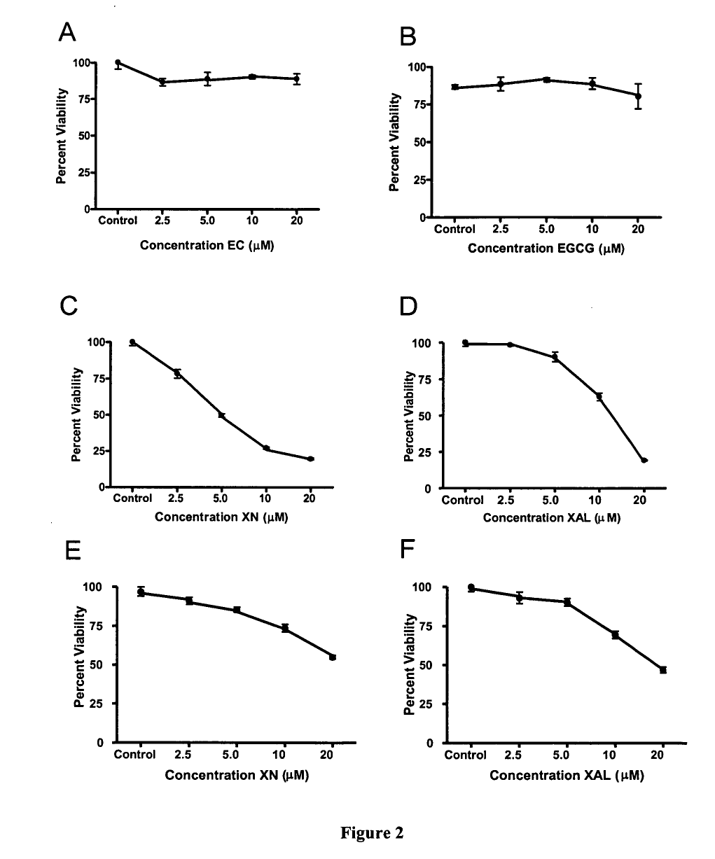

As shown in FIGS. 2(A) and (B), epicatechin and epigallocatechin gallate, two compounds with known antioxidant and anticarcinogenic properties, did not significantly decrease cell viability in BPH-1 cells. In contrast, XN and XAL induced a dose-dependent decrease in number of viable BPH-1 and PC3 cells at 48 h following treatment (FIG. 2(C)-F)). In BPH-1 and PC-3 cells treated with 20 μM XN, viability decreased by 80 and 43%, respectively. In BPH-1 and PC3 cells treated with XAL, viability decreased to 81 and 55%, respectively. A similar effect was observed at 24 h (data not shown) indicating that both XN and XAL significantly inhibit BPH-1 and PC-3 cell viability and proliferation in a time and dose dependent manner.

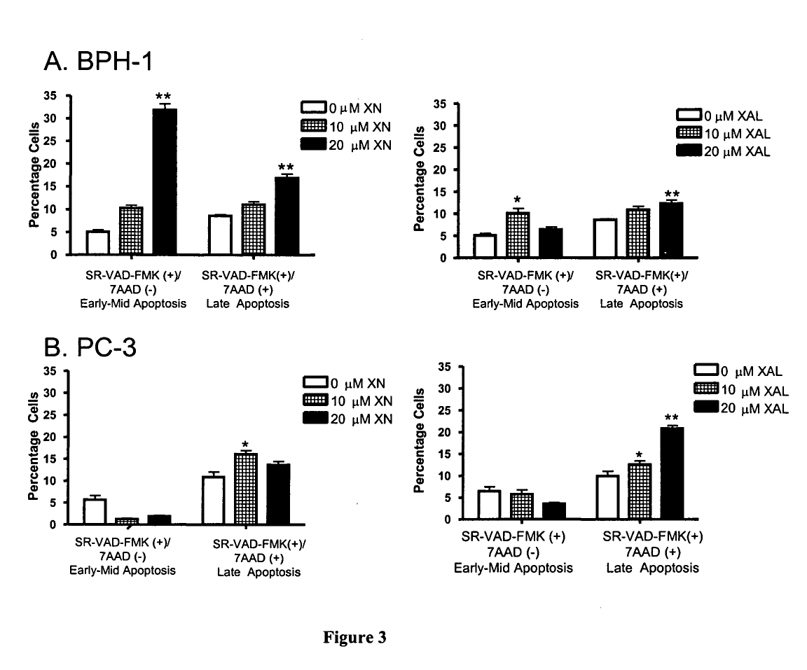

In BPH-1 cells (FIG. 3(A)), treatment with XN and XAL for 48 h resulted in an increase in SR-VAD-FMK stained cells (SR-VAD-FMK(+) 7AAD(−)) indicative of caspase activation without membrane alterations (early to mid stage apoptosis). In addition, there was an increase in doubly stained SR-VAD-FMK(+) 7AAD(+), indicative of cells in late apoptosis (see FIG. 3(A)). As shown in FIG. 3(B), 48 h treatment of PC-3 cells resulted in an increase only in late apoptotic SR-VAD-FMK(+) 7AAD(+) cells.

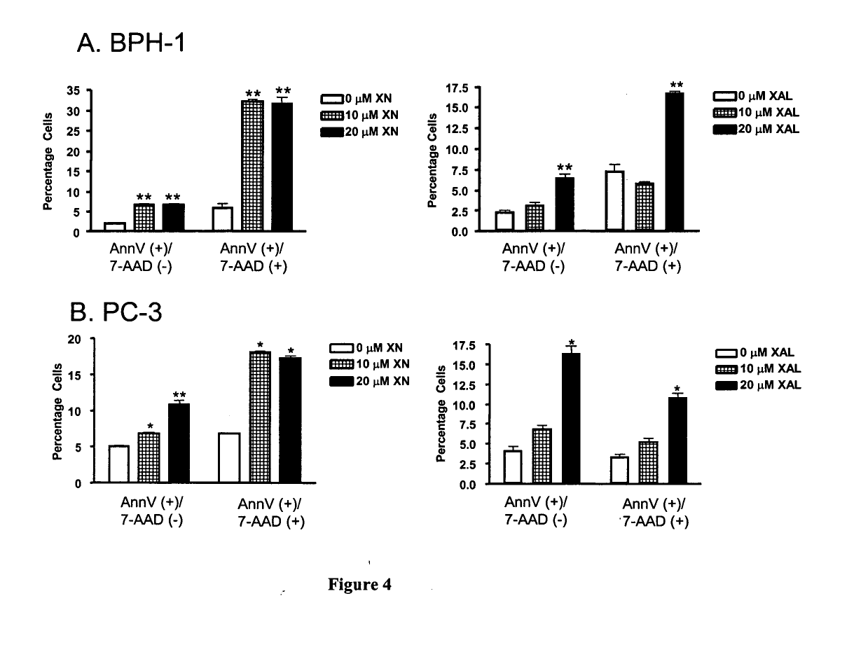

Apoptosis was also quantified by measuring externalization of membrane phosphatidylserine, a characteristic of cells undergoing apoptosis (Martin et al., J. Exp. Med. 182 (1995) 1545-1556). In BPH-1 cells, treatment with 20 μM XN and XAL induced an increase in both Annexin V(+) 7AAD(−) and Annexin V(+) 7AAD(+) (FIG. 4(A)). A similar effect was observed in PC3 cells (FIG. 4(B)).

Table 1 shows XN and XAL mediated cell cycle modifications. BPH-1 cells treated with 10 and 20 μM XN and XAL showed clear accumulation in the S phase, correlating with decreased number of cells in the G1 phase. The cell cycle arrest was also coupled with a concomitant increase in a subG1 peak, an additional indication of apoptosis (Table 1).

TABLE 1
XN and XAL alter cell cycle distribution in BPH-1 Cells

| | Sub G1 (apoptotic) | G1 | S | G2/M |
|---|---|---|---|---|
| Control | 0.870 ± 0.436 | 60.866 ± 0.0689 | 28.040 ± 0.049 | 11.095 ± 0.653 |
| 10 μM XAL | 20.047** ± 0.755 | 38.950** ± 0.995 | 55.283** ± 2.839 | 6.100* ± 1.524 |
| 20 μM XAL | 27.553** ± 1.269 | 45.730 ± 1.376 | 48.391** ± 0.524 | 5.879** ± 1.667 |
| 10 μM XN | 33.204** ± 0.250 | 52.249** ± 0.862 | 41.371** ± 0.568 | 6.380* ± 0.374 |
| 20 μM XN | 28.473** ± 1.182 | 41.196** ± 2.077 | 52.590** ± 2.426 | 6.214* ± 0.367 |

In Table 1, Cells were treated with 0, 10 or 20 μM XN or XAL, as indicated, and harvested after 48 h. Attached and floating cells were fixed in 70% ethanol and stained with propidium iodide, and cell cycle kinetics were examined using the Guava PCA, followed by data analysis with Multi-Cycle software. Results indicate mean (percentage of cells in phase)±SE, n=3. *P<0.05, **P<0.01.

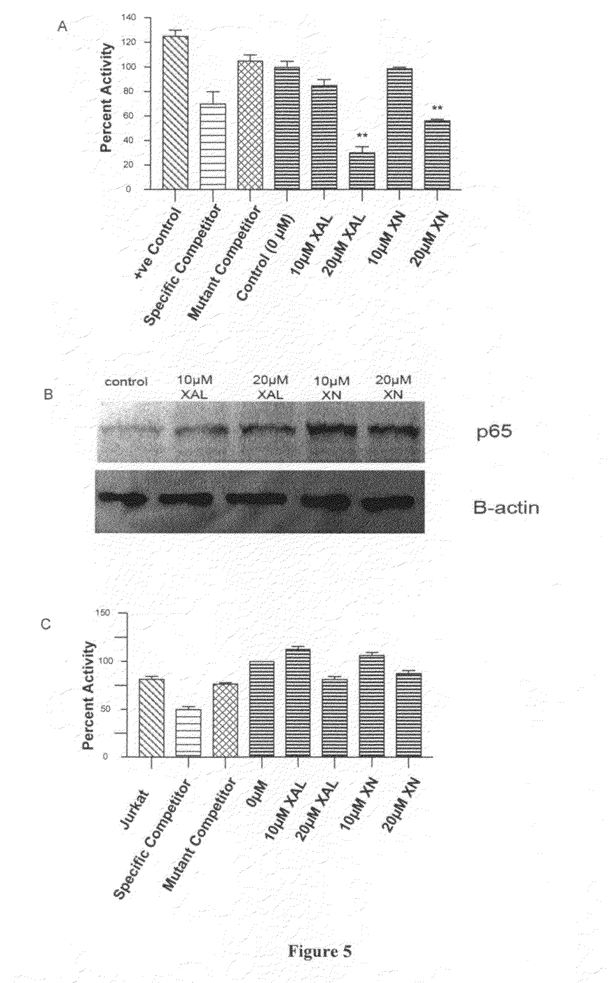

Treatment of BPH-1 cells with 20 μM XN or XAL resulted in a 42% decrease in NFκB activity (see FIG. 5(A)). As shown in FIG. 5(B), NFκB p65 protein levels increased in the nuclear fraction of treated cells.

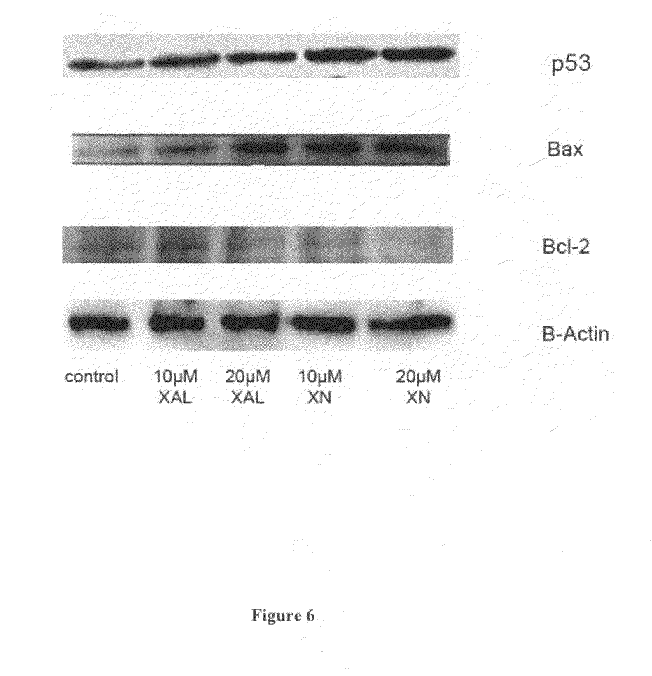

XN and XAL induce apoptosis by examining pro- and anti-apoptotic protein expression through immunoblotting. Increase of pro-apoptotic proteins Bax and p53 was detected in BPH-1 cells treated with 10 and 20 μM XN and XAL (FIG. 6). Treatment of BPH-1 cells with XN resulted in a larger increase in expression of Bax and p53 than did treatment with XAL, perhaps indicating higher potency of XN. The increased expression of pro-apoptotic proteins was coupled with the decreased expression of anti-apoptotic protein Bcl-2 in BPH-1 cells treated with XN and XAL (FIG. 6). In BPH-1 cells, there was a decrease in NFκB activation, despite an apparent increase in p65 nuclear localization in BPH-1 cells (see FIGS. 5(A) and 5(B)).

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A method of treating or preventing prostate cancer in a subject in need of such treatment, said method comprising administering to said subject an effective amount of xanthohumol or xanthohumol analog.

2. The method of claim, wherein said prostate cancer is early stage prostate cancer.

3. The method of claim, wherein said xanthohumol is administered to said subject.

4. The method of claim, wherein said xanthohumol analog is administered to said subject.

5. The method of claim 4, wherein said xanthohumol analog is xanthoaurenol.

6. A method of decreasing cell viability in a prostate cancer cell, said method comprising contacting said cell with xanthohumol or xanthohumol analog.

7. The method of claim 6, wherein said prostate cancer cell is a malignant androgen-independent prostate cancer epithelial cell.

8. The method of claim 6, wherein said xanthohumol is contacted with said prostate cancer cell.

9. The method of claim 6, wherein said xanthohumol analog is contacted with said prostate cancer cell.

10. The method of claim 9, wherein said xanthohumol analog is xanthoaurenol.

11. A method of decreasing cell viability in a benign prostatic hyperplasia cell, said method comprising contacting said cell with xanthohumol or xanthohumol analog.

12. The method of claim 11, wherein said benign prostatic hyperplasia cell is a human benign prostatic hyperplasia epithelial cell.

13. The method of claim 11, wherein said xanthohumol is contacted with said benign prostatic hyperplasia cell.

14. The method of claim 11, wherein said xanthohumol analog is contacted with said benign prostatic hyperplasia cell.

15. The method of claim 12, wherein said xanthohumol analog is xanthoaurenol.

16. A method of increasing apoptosis in a cell, said method comprising contacting said cell with xanthohumol or xanthohumol analog.

17. The method of claim 14, wherein said cell is prostate cancer cell or a benign prostatic hyperplasia cell.

18. The method of claim 14, wherein said xanthohumol is contacted with said cell.

19. The method of claim 14, wherein said xanthohumol analog is xanthohumol is contacted with said cell.

20. The method of claim 17, wherein said xanthohumol analog is xanthoaurenol.

21. A method of treating or preventing benign prostatic hyperplasia in a subject in need of such treatment, said method comprising administering to said subject an effective amount of xanthohumol or xanthohumol analog, thereby treating or preventing benign prostatic hyperplasia in the subject.

22. The method of claim 21, wherein said xanthohumol is administered to said subject.

23. The method of claim 21, wherein said xanthohumol analog is administered to said subject.

24. The method of claim 23, wherein said xanthohumol analog is xanthoaurenol.

25. The method of claim 21, wherein administering to said subject said effective amount of xanthohumol or xanthohumol analog results in treating a symptom of the benign prostatic hyperplasia in the subject.

26. The method of claim 25, wherein said symptom is a male urinary symptom.

27. The method of claim 26, wherein said symptom is incomplete bladder emptying, high urination frequency, urination intermittency, high urination urgency, weak urination stream, or straining to commence urination.

28. The method of claim 25, wherein said xanthohumol is administered to said subject.

29. The method of claim 25, wherein said xanthohumol analog is administered to said subject.

30. The method of claim 27, wherein said xanthohumol analog is xanthoaurenol.
